(19) United States
(12) Patent Application Publication
(10) Pub. No.: US 2024/0002475 A1
(43) Pub. Date: Jan. 4, 2024

(54) COMPOSITIONS AND METHODS FOR RAPID PRODUCTION OF VERSATILE SINGLE DOMAIN ANTIBODY REPERTOIRES

(71) Applicant: The Rockefeller University, New York, NY (US)

(72) Inventors: Brian T. CHAIT, New York, NY (US); Michael P. ROUT, New York, NY (US)

(21) Appl. No.: 18/255,043

(22) PCT Filed: Dec. 3, 2021
(86) PCT No.: PCT/US2021/061741

§ 371 Date: May 30, 2023

Related U.S. Application Data
(60) Provisional application No. 63/120,979, filed on Dec. 3, 2020.

(51) Int. Cl. C07K 16/00; G01N 33/68

(52) U.S. Cl. CPC C07K 16/00; G01N 33/6857; C07K 2317/92; C07K 2317/22; C07K 2317/569

(57) ABSTRACT

Provided are compositions and methods for producing large repertoires of recombinant single domain antibodies with high affinities and specificities against any antigen. Included are methods for making and identifying single domain antibodies produced by camelids, the single domain antibodies themselves, modifications of the nanobodies, expression vectors encoding the nanobodies, cDNAs encoding the nanobodies, cells comprising the expression vectors and/or cDNA, and methods of making the single domain antibodies re-combinantly Antigen-specific single domain antibodies and antigen binding fragments thereof having a Kd for the antigen in a sub-micromolar range are provided. The use of Protein M in isolating Ag-specific HCAbs, and digesting the isolated HCAbs using IdeS protease, is an aspect of this disclosure.

### CROSS REFERENCE TO RELATED APPLICATION

This application claims priority to U.S. provisional patent application No. 63/120,979, filed Dec. 3, 2020, the disclosure of which is incorporated herein by reference.

### STATEMENT REGARDING FEDERALLY SPONSORED RESEARCH

This invention was made with government support under contract nos. GM103314 and P41 GM103314 awarded by the National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy was created on Dec. 2, 2021, has the title “RU_Llama_PCT.txt” and has a file size of 4,096 bytes.

### FIELD

The present disclosure relates generally to novel single domain antibodies and improved methods for making diverse catalogs of such antibodies against any desired antigen(s).

### BACKGROUND

There is a continuing need in biomedicine for reagents such as antibodies that recognize target molecules with high affinity and specificity. When high affinity antibodies are not available, common protein tags such as GFP, FLAG, and myc have been invaluable for many cell biological and biochemical applications. However, most such studies still demand high quality antibodies against these protein tags, particularly when affinity isolation is required. Although monoclonal or polyclonal antibodies remain the primary bait reagents available for these purposes, their large size, limited availability, batch to batch variation, and the frequent non-specific IgG contamination inherent to these reagents have often proved problematic for biochemical or proteomic studies.

An alternative to traditional antibodies has emerged, that of “single domain antibodies,” also referred to as nanobodies. Antibodies from camelids, such as llamas, include a unique subset of immunoglobulins consisting of heavy chain homodimers devoid of light chains. Their variable region (V_{H}H) is the smallest antigen-binding fragment found in the antibody world, and as a single polypeptide chain it is especially suitable for protein engineering. Single domain antibodies are the recombinant minimal-sized, intact antigen-binding domains derived from the V_{H}H region of these heavy-chain antibodies. Unlike monoclonal antibodies, they can be readily produced in large amounts in simple bacterial expression systems Moreover, single domain antibodies are usually extremely stable, can bind antigens with affinities in the nanomolar range, and are smaller in size (approximately 15 kDa) and thereby easier to manipulate genetically as compared with antibody fragments such as ScFvs. However, rapid and robust techniques for the isolation of extensive repertoires of high affinity single domain antibodies have proven elusive—the labor-intensive nature and poor efficiency of current approaches (e.g., phage display) have proven a major bottleneck for the widespread implementation of these reagents explaining why demand for these reagents greatly exceeds supply. The present disclosure meets these and other needs.

### SUMMARY

The present disclosure relates to producing large repertoires of recombinant single domain antibodies with high affinities and specificities against any antigen. Representative and non-limiting examples of single domain antibodies that are aspects of this disclosure, and which demonstrate feasibility of generating large repertoires against any antigen are provided.

Generally the disclosure provides a method for identifying single domain antibodies produced by camelids, the single domain antibodies themselves, modifications of the nanobodies, expression vectors encoding the nanobodies, cDNAs encoding the nanobodies, cells comprising the expression vectors and/or cDNA, and methods of making the single domain antibodies.

In embodiments, the disclosure comprises single domain antibody production by introducing one or more antigens into the camelids, and recombinant methods for producing single domain antibodies that bind with specificity to the antigen(s) (Ag-specific nanobodies). The single domain antibodies that are a subject of this disclosure comprise a heavy chain only IgG class of antibodies (HCAbs). Accordingly, they are comprised of contain heavy chain homodimers, and do not contain antibody light chains. In embodiments, the HCAbs comprise or consist of a single variable domain (V_{H}H) and two constant domains (CH2 and CH3). The single variable domain comprises three complementarity-determining regions (CDRs).

In one aspect the disclosure comprises a method for identifying and/or isolating Ag-specific HCAbs method comprising: i) introducing into a camelid a desired antigen such that a plurality of Ag-specific HCAbs is produced by the camelid; ii) testing lymphocytes obtained from the camelid to determine polynucleotide sequences encoding the variable region (VHH) of a mixed population of HCAbs that includes the plurality of Ag-specific HCAbs and HCAbs that are not specific for the antigen (referred to herein as non-specific HCAbs), and deducing the amino acid sequences of the VHH regions of the Ag-specific HCAbs and non-specific HCAbs in the mixed population from the polynucleotide sequences. In an embodiment, the method further comprises processing a sample from the camelid to separate Ag-specific HCAbs from non-specific HCAbs and determining the amino acid sequences of at least a portion of the VHH regions of the Ag-specific HCAbs, which can optionally be performed with or without proteolytic digestion (i.e., papain digestion) and iv) comparing deduced amino acid sequences of ii) with amino acid sequences of iii) to identify amino acid sequences of ii) that are the same as the amino acid sequences of iii), thereby identifying the Ag-specific VHH regions that are members of the mixed population of HCAbs. In embodiments, at least the comparing step is performed by a computer and using an algorithm, and may include a microprocessor implemented comparison of the amino acid sequences, such as a microprocessor implemented comparison of the measured tandem mass spectra of ii) and the calculated tandem mass spectra of iii). In embodiments, the described optional protease digestion is performed. In embodiments, the protease used is IdeS protease, which has been demonstrated to be superior in the described method relative to trypsin digestion.

Methods of the disclosure are suited for determining large numbers of sequences. In one embodiment, determining the polynucleotide sequences of ii) comprises generating and sequencing a plurality of cDNA sequences that encode at least 10,000 unique VHH regions. In certain aspects the disclosure includes separating Ag-specific antibodies or antigen-binding fragments thereof from the non-specific antibodies by affinity purification of the Ag-specific antibodies using the antigen as an affinity capture agent.

In embodiments, the disclosure includes modifications of the described method, which are further described by illustrative protocols in the Examples, said protocols being suitable for identifying Ag-specific HCAbs that are described herein. In an embodiment, the disclosure provides a method for identifying single domain antibodies that bind with specificity to an antigen (heavy chain only IgG class of antibodies (Ag-specific HCAbs)), the method comprising: i) introducing into a camelid an antigen such that a plurality of Ag-specific HCAbs is produced by the camelid; ii) testing lymphocytes obtained from the camelid to determine polynucleotide sequences encoding the variable region (VHH) of a mixed population of HCAbs that includes the plurality of Ag-specific HCAbs and HCAbs that are not specific for the antigen (non-specific HCAbs), and deducing the amino acid sequences of the VHH regions of the Ag-specific HCAbs and non-specific HCAbs in the mixed population from the polynucleotide sequences; iii) processing a sample from the camelid to separate Ag-specific HCAbs from non-specific HCAbs and determining the amino acid sequences of at least a portion of the VHH regions of the Ag-specific HCAbs; and iv) comparing deduced amino acid sequences of ii) with amino acid sequences of iii) to identify amino acid sequences of ii) that are the same as the amino acid sequences of iii), thereby identifying the Ag-specific VHH regions that are members of the mixed population of HCAbs. The disclosure includes adapting the described method in step iii) by separating the Ag-specific HCAbs by removal of non-HCAb antibodies using Protein M, isolating Ag-specific HCAbs, digesting the isolated HCAbs using IdeS protease, separating digested fragments comprising segments of the VHH regions by gel electrophoresis to obtain separated fragments of the VHH regions from the gel, and using the separated segments of the VHH regions to determine the amino acid sequences of at least a portion of the Ag-specific HCAbs.

In certain embodiments, lymphocytes, such as B plasma cells, are obtained from bone marrow of the camelid for use in the identification of the VHH regions.

In another aspect the disclosure further comprises providing and introducing

distinct expression vectors encoding distinct Ag-specific single domain antibodies into host cells, wherein the single domain antibody sequences are designed based on the deduced Ag-specific VHH regions allowing expression of the distinct Ag-specific single domain antibodies from the host cells, separating the Ag-specific single domain antibodies from the host cells, and testing the Ag-specific single domain antibodies for binding to the antigen.

In embodiments, any one or any combination of Ag-specific single domain antibodies or antigen-binding fragments therefore have a Kd for the antigen in a sub-micromolar range.

### DESCRIPTION OF THE FIGURES

FIG. 1. Schematic overview of representative method.

### DETAILED DESCRIPTION

Unless defined otherwise herein, all technical and scientific terms used in this disclosure have the same meaning as commonly understood by one of ordinary skill in the art to which this disclosure pertains.

Every numerical range given throughout this specification includes its upper and lower values, as well as every narrower numerical range that falls within it, as if such narrower numerical ranges were all expressly written herein.

The disclosure includes all steps and compositions of matter described herein in the text and figure of this disclosure, including all such steps individually and in all combinations thereof, and includes all compositions of matter. The disclosure includes all polynucleotide sequences, their RNA or DNA equivalents, all complementary sequences, and all reverse complementary sequences. If reference to a database entry is made for a sequence, the sequence is incorporated herein by reference as it exists in the database as of the filing date of this application or patent. Sequences that are 80.0-99.9% identical, inclusive, and including all numbers to the first decimal point there between, to any nucleotide or amino acid sequence are encompassed by this disclosure.

The amino acid sequences of all antibody sequences, including all single domain antibody sequences, that are described in PCT publication WO 2015/200626, are incorporated herein by reference, as are the laboratory names given to the antibodies.

The present disclosure provides in various embodiments methods that are useful for generating large repertoires of readily expressible recombinant single domain antibodies with high affinities and specificities against a given antigen. In general the disclosure comprises methods for identifying single domain antibodies produced by camelids in response to introducing one or more antigens into the camelids, and recombinant methods for producing single domain antibodies that bind with specificity to the antigen(s) (Ag-specific nanobodies). The single domain antibodies referenced herein are a component of the heavy chain only IgG class of antibodies (HCAbs), and thus contain heavy chain homodimers and do not contain antibody light chains. HCAbs comprise a single variable domain (V_{H}H, which are typically between 110-150 amino acids in length) and two constant domains (CH2 and CH3). The single variable domain comprises three complementarity-determining regions (CDRs).

In one aspect the method comprises the steps: i) introducing into a camelid an

antigen such that a plurality of Ag-specific HCAbs is produced by the camelid; ii) testing lymphocytes obtained from the camelid to determine polynucleotide sequences encoding the variable region (V_{H}H) of a mixed population of HCAbs that includes the plurality of Ag-specific HCAbs as well as HCAbs that are not specific for the antigen (non-specific HCAbs), and deducing the amino acid sequences of the V_{H}H regions of the HCAbs in the mixed population from the polynucleotide sequences; iii) processing a sample from the camelid to separate Ag-specific HCAbs from non-specific HCAbs and determining the amino acid sequences of at least a portion of the V_{H}H regions of the Ag-specific HCAbs; and iv) comparing deduced amino acid sequences of ii) with amino acid sequences of iii) to identify amino acid sequences of ii) that are the same as the amino acid sequences of iii), thereby identifying the Ag-specific V_{H}H regions that are members of the mixed population of HCAbs. In embodiments, the disclosure includes providing recombinant expression vectors which encode polypeptides that comprise the Ag-specific V_{H}H regions from members of the mixed population of HCAbs, in vitro cell cultures which comprise such expression vectors, methods of making such expression vectors and cell cultures, and methods of producing polypeptides which comprise the Ag-specific V_{H}H regions.

It is expected that any camelid, meaning any member of the biological family Camelidae, can be used to generate the HCAbs described herein. In embodiments, the camelid is selected from camels, alpacas and llamas.

We demonstrate various embodiments of the present disclosure using illustrative examples. These examples include but are not limited to recombinant production of a repertoire of unique single domain antibodies that were initially identified by immunizing a llama with green fluorescent protein (GFP). The single domain antibodies exhibit K_{d} values into the sub-nanomolar range. By utilizing the diversity of this single domain antibody population and mapping their binding epitopes, we were also able to design ultra-high affinity dimeric nanobodies, with K_{d}s down to sub-pM. Similar repertoires were generated against a variety of biological targets, including human cell surface receptors, nucleoporins, and viral proteins. It will thus be recognized by those skilled in the art that aspects of the present disclosure are suitable for production of high affinity capture reagents for a multitude of biomedical applications. It is expected that various aspects of the disclosure will produce compositions that are useful for prophylaxis and/or therapy of disorders that are associated with the presence of one or more antigens, as well as for use in various diagnostic and medical imaging techniques.

The methods of this disclosure can be used to produce, identify, clone and express HCAbs that are specific for any antigen that can stimulate HCAb production in a camelid. In embodiments, the antigen is a protein or peptide antigen. In embodiments, the antigen is a polysaccharide or nucleic acid, or their derivatives. In an embodiment the antigen is any molecule, compound or composition that can stimulate antibody production. In embodiments, the antigen is a cell surface receptor. In embodiments, more than one antigen can be introduced, resulting in production of a diverse ensemble of HCAbs. Up to 13 antigens have been simultaneously coinjected for immunization, with no loss of HCAb response. In embodiments, the antigen(s) which is used to the stimulate the HCAbs can be expressed by any cell type, or by a virus. In embodiments, the antigen is expressed by a cancer cell, or by an infectious agent, such as an infectious microbe that is associated with infections in humans, or non-human animals, or both. In embodiments the antigen is a component of or is an allergen.

In embodiments the antigen is component of or is a toxin. In embodiments the antigen is a component of a tag used to purify fusion proteins which comprise the tag. In an embodiment the antigen is a receptor, such as a cell surface receptor. The antigen may be well characterized, or may be unknown, such as by being part of a multimeric complex, so long as the complex can be used to capture complex-specific HCAbs as generally outlined above and according to various embodiments of this disclosure. Crude mixtures, including but not necessarily limited to cell lysates and other mixtures, could also be used provided the antigen is ultimately determined and used according to the methods of this disclosure to identify V_{H}H regions.

In order to stimulate production of the HCAbs, the antigen or a composition comprising it can be introduced into the camelid using any suitable technique. Some non-limiting examples include oral, parenteral, subcutaneous, intraperitoneal, intrapulmonary, and intranasal. Parenteral infusions include intramuscular, intravenous, intraarterial, intraperitoneal, and subcutaneous administration. The administration can include more than one antigen if desired, meaning distinct antigens, peptides, proteins, and/or carbohydrate antigens can be administered. The composition comprising the antigen can include other components, such as pharmaceutically/veterinarially acceptable carriers, and adjuvants. In particular embodiments described herein and used to demonstrate aspects of this disclosure, animals were immunized with 5 mg of antigen, prepared with Complete Freund's Adjuvant. This was followed by three booster immunizations of 5 mg of antigen, prepared with Incomplete Freund's Adjuvant. Booster immunizations were performed 21, 42, and 62 days after the first immunization. Immunizations could alternatively be performed with smaller amounts of antigen, from 0.1 mg to 5 mg. Test serum bleeds were obtained 52 days after the first immunization, and the animal's immune response was assessed by determining the specific activity of this serum against the antigen. A production serum bleed and bone marrow aspirate were obtained 74 days after initial immunization.

Once the camelid has produced Ag-specific HCAbs, samples are obtained for analysis as generally outlined above. For use in determining sequences encoding the non-specific and Ag-specific HCAbs the sample can comprise any lymphocytes that comprise DNA sequences encoding the HCAbs. In embodiments, the sample comprises plasma cells, (i.e., plasma B cells and memory B cells). In embodiments, the sample comprises bone marrow. In embodiments, the sample comprises or consists of a bone marrow aspirate. In embodiments, the sample comprises mononuclear cells that are separated from a bone marrow sample to provide a cell composition that is enriched for plasma cells.

In embodiments, the lymphocytes are processed to separate mRNA or total RNA for use in generating a cDNA library. As is well known in the art, generating a cDNA library comprises reverse transcription of the RNA to obtain DNA templates from which the V_{H}H variable regions encoding the plurality of Ag-specific HCAbs as well as the non-specific HCAbs are amplified. The cDNA library can be produced using any suitable techniques. In embodiments, a nested PCR approach is used to amplify the plurality of Ag-specific and non-specific HCAbs. In embodiments, the cDNA library comprises as many as 10⁷ unique V_{H}H coding sequences. The DNA sequences of the PCR amplicons from the cDNA library are then determined using any suitable technique. In general, high-throughput DNA sequencing methods are used, such as so-called deep sequencing, massively parallel sequencing and next generation sequencing, which are well known techniques and are offered commercially by a number of vendors. In embodiments, the amplified cDNAs are sequenced by high-throughput 454 sequencing, MiSeq sequencing or NovaSeq sequencing. In embodiments, the high-throughput sequencing results in 800,000 to 150,000,000 unique reads. Determining the sequences in this manner provides a catalog of sequences encoding the V_{H}H variable regions of the Ag-specific and non-specific HCAbs. From this catalog, the amino acid sequences of these V_{H}H variable regions are deduced (translated in silico), thus providing a catalog of V_{H}H variable regions, some of which are specific for one or more epitopes present the antigen administered to the camelid, and many of which are not specific for the antigen. In embodiments, the translated reads can be subjected to computational analysis, which can include but is not necessarily limited to in silico protease digestion, the results of which can be stored in a text file or indexed in a searchable peptide database stored on a computer or other digitized media. The text file or searchable database can be configured to account for a variety of parameters, such as the distinct sequences of in silico digested peptides, the number of cDNA sequencing reads that relate to each of those peptides sequences, and the sequences of the complementarity determining regions (CDR1, CDR2 and CDR3), and the framework regions if desired.

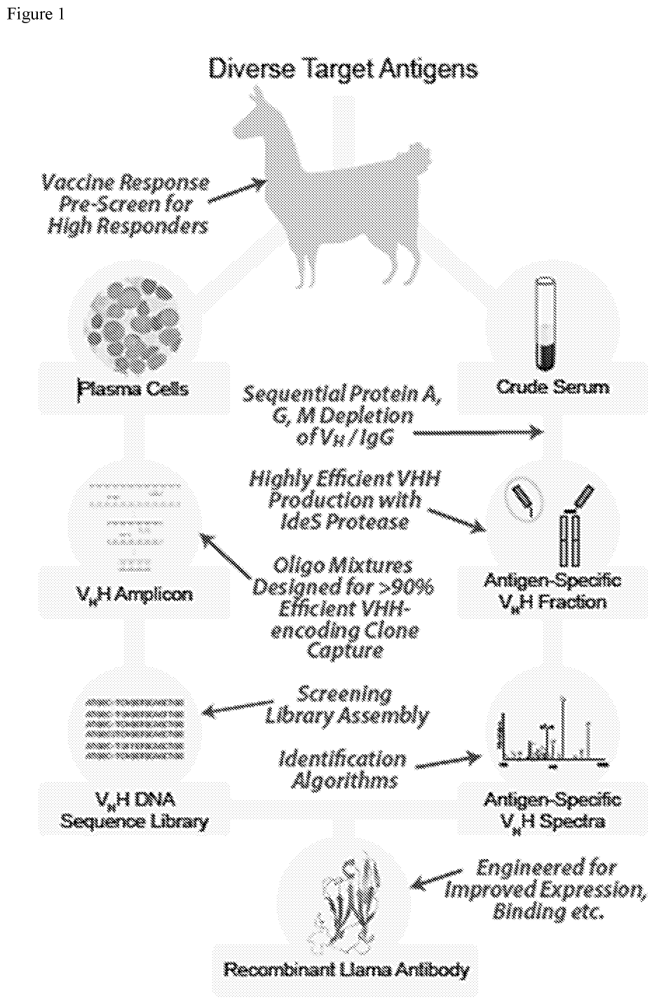

In order to determine distinct V_{H}H variable region sequences that are responsible for specificity for the antigen, a sample from the camelid is processed to separate Ag-specific HCAbs from non-specific HCAbs. In general any suitable sample comprising HCAbs can be used for this purpose. In embodiments, one or a series of serum samples is obtained and processed, for example, to obtain an IgG fraction, such as a fraction that is enriched for V_{H}H IgG. In embodiments, sequential purification of serum over immobilized Protein G and Protein A, with selective elution at pH 3.5 to 4.0, results in separation of HCAb that comprises Ag-specific and non-specific IgG components. The HCAb is further purified by incubation with immobilized Protein M, capturing residual light-chain associated IgG (FIG. 1). The V_{H}H IgG is then processed using an affinity purification approach, which involves use of the immunizing antigen as a capture agent.

Any suitable affinity capture approach can be used and will generally comprise fixing the antigen to a solid substrate, such as a bead or other material, and mixing the Ag-specific HCAbs and non-specific HCAbs (i.e., separated V_{H}H IgG) with the substrate-fixed antigen such that only the Ag-specific HCAbs are retained on the substrate-fixed antigen. This process yields a composition comprising Ag-specific HCAbs reversibly and non-covalently bound to the substrate-fixed antigen. In embodiments, the antigen-specific HCAbs can be treated to remove the Fc portion, such as by exposure to papain, which consequently provides a composition that is enriched with antigen-specific V_{H}H fragments bound to the capture agent. The V_{H}H fragments can be eluted from the capture agent and purified if desired using any suitable approach. Thus, a composition comprising isolated and/or purified V_{H}H fragments is provided for amino acid sequence analysis. Further, while papain digestion is a functional option, it sometimes produces variable results that result from using a protease digestion step, such as with batch variation in the protease, differential sensitivity, and the like. However, we have determined that, because mass spectrometry has the necessary sensitivity, dynamic range and specificity, the protease digestion step can avoided if desired, and mass spectrometry used instead to directly identify the specific heavy chain only IgGs. A preferred option is the use of a site-specific IgG protease such as S. pyogenes IdeS. Unlike papain, which is highly prone to non-specific digestion of protein, IdeS is highly specific for IgG and will cleave off Fc without affecting the immobilized antigen (FIG. 1). This avoids any risk of overdigestion of either antigen or bound VHH, whereas papain cleavage must be precisely titrated.

The amino acid sequence analysis of the VHH fragments can be performed using any appropriate technique. In an embodiment, mass spectrometric (MS) analysis of the Ag-specific V_{H}H regions is performed and is interpreted such that the CDR sequences or portions thereof are determined. In an embodiment, a computer/microprocessor implemented comparison of the amino acid sequences determined from the Ag-specific V_{H}H regions and the amino acid sequences deduced from the cDNA analysis is performed so that matching sequences can be identified, thus identifying Ag-specific V_{H}H amino acid sequences. In another embodiment, the fragment masses of the amino acid sequences deduced from the cDNA analysis are calculated and compared to tandem mass spectra of the Ag-specific V_{H}H regions. In embodiments, this comparison can include data and rankings related to the MS coverage of complementarity determining regions, which in embodiments includes all of CDR1, CDR2 and CDR3 sequences, mass spectral counts, and expectation values of peptide sequences that match the cDNA and MS sequence data. Based on this analysis, the sequence of Ag-specific V_{H}H regions can be identified. Incorporating Protein M clean-up, IdeS digestion, hinge-specific primers, and optimized MS sample preparation and analysis increased typical number of identified nanobody candidates from 10-100 to 100-1000.

Once the amino acid sequence of Ag-specific V_{H}H regions are in hand they can be introduced into expression vectors so that Ag-specific single chain antibodies can be made recombinantly for further testing, or for use in a wide variety of other methods. In this regard, any suitable expression vector and protein expression system can be used. The expression vector is not particularly limiting other than by a requirement for the Ag-specific single chain antibodies to be driven from a suitable promoter, and many suitable expression vectors and systems are commercially available. In embodiments the expression systems can be eukaryotic or prokaryotic expression systems, such as bacterial, yeast, mammalian, plant and insect expression systems. In general the expression vector will include at least one promoter driving expression of the single chain antibodies mRNA from its gene, and may include other regulatory elements to effect and/or optimize expression of the inserted single chain antibody's coding region. The promoter can be a constitutive or inducible promoter. Suitable expression vectors can thus comprise prokaryotic and/or eukaryotic promoters, enhancer elements, origins of replication, selectable markers for use in maintaining the expression vectors in the desired cell type, polycloning sites, and may encode such features as visually detectable markers. More than one promoter can be included, and more than one single chain antibody can be encoded by any particular expression vector, if desired. The expression vectors can also be adapted to express single chain antibody-fusion proteins. The fusion proteins can include any other amino acid sequence that would be desirable for expressing in the same open reading frame as the single chain antibody. The single chain antibody sequence can be configured N-terminal or C-terminal to the fused open reading frame, depending on the particular fusion protein to be produced. In one embodiment, the protein expression is a bacterial system. In embodiments, the Ag-specific single chain antibodies identified and produced recombinantly according to this disclosure can exhibit a Kd in a sub-micromolar range, such as a nM range. All cDNA sequences encoding single chain antibodies identified by the methods of this disclosure are encompassed within its scope. The polynucleotide sequence encoding the Ag-specific single chain antibodies can be optimized, such as by optimizing the codon usage for the particular expression system to be used. Further, and as described briefly above, the Ag-specific single chain antibody protein that is expressed can be configured such that it is a component of a fusion protein. Such fusion proteins can include components for use in facilitating purification of the Ag-specific single chain antibody from the expression system, such as a HIS or FLAG tag, or can be designed to impart additional function to the single chain antibody, such as by providing a detectable label or cytotoxic moiety, or to improve solubility, secretion, or any other function. In various embodiments, the Ag-specific single chain antibodies and/or fragments can be conjugated to a chemotherapeutic agent to enable localization of the chemotherapeutic agent to cells which express the antigen. Chemotherapeutic agents useful in the generation of such Ag-specific single chain antibodies conjugates include but are not necessarily limited to enzymatically active toxins and fragments thereof. In another embodiment, the Ag-specific single chain antibody and/or fragments thereof may be conjugated to a detectable label, such as a radioactive agent or a fluorescent moiety, for use in labeling cells or tissues in medical imaging techniques, or for targeted killing of cells which express the antigen. In embodiments, any of variety of radioactive isotopes are available for conjugating to Ag-specific single chain antibodies such that cells to which the Ag-specific single chain antibodies bind may be imaged or selectively destroyed. For selective destruction of cells expressing the antigen, the Ag-specific single chain antibodies that recognize the antigen can be conjugated to a highly radioactive atom, such as In¹¹¹, At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu. When the Ag-specific single chain antibodies and/or fragments thereof are used for identifying cells expressing the antigen they can comprise a radioactive atom for scintigraphic studies, for example Tc^{99m} (metastable technetium-99), I¹²³, or a spin label for nuclear magnetic resonance (NMR) imaging (also known as magnetic resonance imaging, or “MRI”), such as I¹²³, I¹³¹, I¹²⁴, F¹⁹, C¹³, N¹⁵, O¹⁷ or Gadlinium (III) or Manganese (II).

In embodiments, the Ag-specific HCAbs can be partially or fully humanized for use in prophylaxis and/or therapy of a condition that is positively associated with the presence of the antigen. In general, humanization involves replacing all or some of the camelid derived framework and constant regions of Ag-specific single chain antibodies with human counterpart sequence, with the aim being to reduce immunogenicity of the Ag-specific single chain antibodies in therapeutic applications. In some instances, the FR residues of the human immunoglobulin are replaced by corresponding non-human residues. In general, a humanized

Ag-specific single chain antibody will comprise substantially all of at least one, and typically two, variable domains, in which all or substantially all of the hypervariable loops correspond to those of the non-human, camelid immunoglobulin and all or substantially all of the FRs are those of a human immunoglobulin sequence.

In embodiments, the disclosure includes making single chain antibody homo- and hetero dimers, trimers, tetramers, and the like. For example, a single single chain antibody heterodimer can have specificity for two distinct epitopes on the antigen, or it could have specificity for distinct epitopes on two different antigens. After identification, two such HCAb candidates can be genetically fused, joined with a peptide linker sequence.

In another aspect, the disclosure includes use of single chain antibodies

generated as described herein to map epitopes on the antigen that was used to raise the HCAb in the camelid. This aspect comprises binding one or more distinct single chain antibodies to the antigen and determining the location of the single chain antibody binding on the antigen, thereby identifying the epitope or amino acids which are comprised by the epitope. In embodiments the location of the single chain antibody can be determined by NMR. Epitope mapping provided by the disclosure are useful for instance, for determining fragments of antigens that comprise epitopes that are particularly effective in stimulating an antibody response, and accordingly could be used to generate a robust antibody response in a human or non-human animal, thereby providing optimal vaccine candidates.

This disclosure encompasses each and every amino acid sequence described herein, and each and every polynucleotide encoding the amino acid sequences. The amino acid sequences include fragments of at least 4, 5, 6, 7, 8, 9, 10, 11, 12, 13, 14, or 15, or more, contiguous amino acids of any amino acid sequence disclosed herein. In embodiments the disclosure encompasses amino acid fragments that comprise or consist of one or more complementarity determining regions (CDRs). Recombinant expression vectors encoding one or more polypeptides comprising or consisting of one or more of the CDRs are included in the disclosure. Any suitable expression vector can be used for such expression and many are known in the art and/or are commercially available and can be adapted for use according to the instant disclosure. The disclosure includes cells and cell cultures comprising the expression vectors, methods of making such cells and cell cultures comprising introducing such an expression vector into cells, methods of producing single domain antibodies or antigen-binding fragments thereof by allowing expression of the expression vector in the cells and separating the single domain antibodies or fragment thereof from the cells. The disclosure includes isolated single domain antibodies and antigen-binding fragments thereof, and modified single domain antibodies and antigen-binding fragments thereof, such as fusion proteins and protein conjugates, wherein the single domain antibodies and antigen-binding fragments are covalently attached to a chemical moiety. The disclosure also includes one or more cDNAs encoding the single domain antibodies or antigen-binding fragments thereof.

The following Example is presented to illustrate the embodiments of the disclosure. It is not intended to be limiting in any manner.

### EXAMPLE

### Strategy for Single Domain Antibody Identification

The described approach to single domain antibody discovery involves in part a

on bottom-up MS identification of affinity-purified V_{H}H antibodies isolated from an individual llama, in correlation with a DNA sequence database generated from the same animal (FIG. 1). The approach represents a novel pipeline for single domain antibody production where each stage has been h optimized.

After a series of standard immunizations, we collect both serum samples

and bone marrow aspirates (FIG. 1). Plasma cells are enriched in marrow aspirates compared to blood. They produce high affinity antibodies and express elevated levels of immunoglobulin RNA and therefore are a superior source for generating cDNA libraries. Importantly, we do not create expression libraries, and thus remove the need for efficient exogenous expression, folding, and presentation of the clones; rather, we take advantage of high-throughput sequencing to produce large sequence databases from cDNA, covering the V_{H}H variable region repertoire produced by the immunized animal. In contrast to conventional antibodies, single domain antibody elucidation does not require the pairing of heavy and light chains, allowing for easy generation of comprehensive sequence libraries.

In parallel, native polyclonal antigen-specific antibodies are obtained from serum isolated directly from the immunized animal. Affinity purification can be adjusted to generate fractions of antibodies with the highest affinity and specificity. We also take advantage of new advances in mass spectrometry that enable the identification of hundreds of proteins from a single sample, such as the presently considered enriched fractions of V_{H}H antibodies (FIG. 1). We built a user-friendly MS protocol and interpretive program that allows the rapid and accurate identification of the V_{H}H sequences. The high efficiency of this method allows us to then directly produce codon optimized single domain antibody expression constructs in order to enable high expression and facile purification. Finally, we use a straightforward screen to determine those recombinant single domain antibodies that express and bind well to the antigen.

Testing the approach on two high utility antigens. To generate a single domain antibody repertoire of maximal utility, we chose the GFP and mCherry tags for first target antigens, due to their central roles in cell biological studies and because so many cell lines and transgenic animals carry these proteins. Further, while these fluorescent proteins have a broadly similar beta barrel structure, they are in fact significantly evolutionarily divergent, being from jellyfish and coral species (separated by ˜700 million years), and have only 26% sequence similarity, making for distinct immunogens. After immunization of individual llamas with these antigens and confirmation of an immune response, we serially fractionated crude serum bleeds to obtain exclusively V_{H}H-containing heavy chain antibodies, taking advantage of the differential specificity of Protein A and Protein G for V_{H}H-containing heavy chain antibodies versus conventional antibodies. To eliminate excess standard VH IgG, immobilized Protein M was used as an additional purification step when necessary, depleting light-chain associated IgG. V_{H}H-containing heavy chain antibody fraction was then affinity purified over antigen-coupled resin, and washed with MgCl₂ at various stringencies, from 1 M to 3.5 M, allowing the isolation of antibodies enriched for different levels of affinity. The antibodies were then digested with papain or higher specificity IdeS protease on-resin to cleave away the constant regions and leave behind the desired minimal V_{H}H variable region fragments. Finally, the antigen-bound V_{H}H fragments were eluted and separated by SDS-PAGE, allowing the purification of the ˜15 kDa or ˜20 kDa V_{H}H fragments away from Fab fragments derived from contaminating conventional antibodies as well as Fc fragments (both ˜25 kDa), and undigested antibodies (˜50 kDa). The gel purified bands were then trypsin-digested and analyzed by liquid chromatography-MS and MS/MS. We recovered the highest affinity V_{H}H fragments by using the highest stringency washes, which also significantly decreased the complexity of the eluted sample, aiding MS analysis when large repertoires of antibody were bound. To create an animal-specific antibody sequence database, lymphocyte mRNA samples from individual immunized llamas were obtained for high-throughput sequencing. Mononuclear cells were isolated from bone marrow aspirates, enriching for long-lived antibody secreting plasma cells Total RNA from these cells was reverse transcribed, and a nested PCR was performed to specifically amplify sequences encoding the V_{H}H variable regions. This PCR product was then sequenced by high-throughput Roche 454 or Illumina MiSeq or NovaSeq sequencing, resulting in approximately 0.8 million, 5 million, or 150 million unique reads, respectively. These reads were translated, filtered and trypsin-digested in silico to create a searchable peptide database for MS analysis. To illustrate one aspect of this process, for GFP cloning in a first step, CALL001 (5′-GTCCTGGCTGCTCTTCTACAAGG-3′ SEQ ID NO:1)) and CALL002 (5′-GGTACGTGCTGTTGAACTGTTCC-3′ SEQ ID NO:2) primers were used to amplify the IgG variable domain into the CH2 domain (Conrath et al, #26). The approximately 600-750 bp band from VHH variants lacking a CH1 domain was purified on an agarose gel. Next, VHH regions were specifically reamplified using framework 1- and 4-specific primers with 5′ 454 adaptor sequences: 454-VHH-forward (5′-CGTATCGCCTCCCTCGCGCCATCAGATGGCT[C/G]A[G/T]GTGCAGCTGGTGGAGTC TGG-3′ (SEQ ID NO:3)) and 454-VHH-reverse (5′-CTATGCGCCTTGCCAGCCCGCTCAG GGAGACGGTGACCTGGGT-3′ (SEQ ID NO:4)). The approximately 400 bp product of this reaction was gel purified, then sequenced by high-throughput 454 sequencing, resulting in approximately 800,000 unique reads. For MiSeq or NovaSeq sequencing, equivalent primers with 12 random nucleotides at the 5′ ends were used instead. Alternative primers were separately designed to recognize a greater proportion of V_{H}H sequences, annealing in the leader and hinge regions flanking the V_{H}H domain. Forward primers 6N_CALL001 5′-NNNNNNGTCCTGGCTGCTCTTCTACAAGG-3′ (SEQ ID NO:5) and 6N_CALL001B 5′-NNNNNNGTCCTGGCTGCTCTTTTACAAGG-3′ (SEQ ID NO:6) and reverse primers 6N_VHH_SH_rev 5′-NNNNNNCTGGGGTCTTCGCTGTGGTGC-3′ (SEQ ID NO:7) and 6N_VHH_LH_rev 5′-NNNNNNGTGGTTGTGGTTTTGGTGTCTTGG G-3′ (SEQ ID NO:8) were used for these PCRs, with 6 random bases (N) added to aid Illumina MiSeq or NovaSeq sequencing.

Because most proteins differ from each other throughout their sequences, they can be readily distinguished by MS-based database sequence searches. However, the identification of specific V_{H}H sequences is more challenging because they comprise in large part highly conserved framework regions. Moreover, rather than searching well-established databases, a V_{H}H cDNA database must be generated for each immunized animal. To deal with both challenges, we developed a bioinformatic pipeline to identify the highest probability matches from a large pool of related V_{H}H sequences (Llama Magic software). In this pipeline, V_{H}H sequences were ranked by a metric based on MS/MS sequence coverage of complementarity determining region 3 (CDR3, the most diverse V_{H}H region) as well as CDR1 and CDR2 coverage, total V_{H}H coverage, sequencing counts, mass spectral counts, and the expectation values of matched peptides. Preliminary attempts to identify V_{H}H sequences solely by their CDR3 regions revealed that identical CDR3 sequences are frequently shared between multiple distinct V_{H}H sequences, with diverse CDR1 and CDR2 sequences. It is likely that this is a result of somatic gene conversion, in which, after V(D)J recombination, secondary recombination occurs between upstream V gene segments and already rearranged V(D)J genes. While this mechanism has not previously been reported in llamas, a number of mammals do exhibit such recombination, including rabbit, swine, and bovine species. The described automatic ranking pipeline, coupled with careful manual inspection, overcame these issues and provided us 44 high-probability hits against GFP, classified as LaG (Llama antibody against GFP) 1-44, which we subjected to further screening. As the additional test antigen, a smaller subset of eight clones was chosen for follow up (LaM 1-8) for mCherry.

Codon optimized genes for these hits were synthesized and cloned into a bacterial expression vector. After expression, lysates were passed over antigen-coupled resin to identify single domain antibodies that displayed both robust expression and high, specific affinity. From this pilot screen, we found 25 specific anti-GFP single domain antibodies out of 44 tested, and 6 anti-mCherry clones out of the 8 screened. Phylogenetic analysis of the verified single domain antibodies revealed significant sequence diversity among these clones. While not directly analogous, the extremely high success rate of this single screening step (57-75%) is very favorable in comparison to the final panning and selection steps of phage display. The affinity of these 25 GFP-binding single domain antibodies and 6 anti-mCherry single domain antibodies was further assessed by either surface plasmon resonance (SPR) or in vitro binding assays with immobilized single domain antibodies. For the larger repertoire of anti-GFP clones, these experiments revealed a wide range of affinities, with K_{d}s from 0.5 nM to over 20 μM, and identified 16 single domain antibodies with very high affinity binding (≤50 nM). The SPR data also showed wide ranges of association and dissociation rates, indicating differences in binding kinetics. As they were derived from a smaller number of high-confidence candidates, the K_{d}s of the six anti-mCherry single domain antibodies were consistently strong, ranging from 0.18 nM to 63 nM. After fully optimized this methodology, additional anti-GFP single domain antibodies were identified with an 80% hit rate (16 out of 20 candidates). These antibodies included several with higher affinity, with K_{d}s as low as 30 pM. Many diverse antigens have shown similar results in terms of repertoire size and affinity.

### Specificity and Efficacy of Recombinantly Produced Nanobodies

We performed a variety of experiments to assess the utility of single domain antibodies in affinity capture and subcellular localizations experiments. Thus, affinity capture experiments were performed on endogenous GFP- and mCherry-tagged proteins in yeast and human cells. All 25 positive GFP binders were used for the isolation of GFP-tagged Nup84, a structural nuclear pore complex component, in budding yeast. We plotted each LaG's observed K_{d} against a quantification of either signal to background or yield from a Nup84-GFP affinity capture. Almost all LaGs were able to pull down detectable amounts of Nup84-GFP and its associated proteins, and many performed as well or better than our best affinity-purified polyclonal antibodies. Similarly, while the single commercially available GFP-Trap® anti-GFP single domain antibody (ChromoTek GmbH) has a low reported K_{d} of 0.59 nM, the performance of the highest-affinity LaGs, as judged by specificity and particularly yield in these pullouts, was comparable or in some cases better. Generally speaking though, a strong correlation is seen between low K_{d} and both high signal to background and high yield. This correlation between yield and K_{d} is broadly consistent with the relationship theoretically predicted for the percentage of a low abundance target bound in solution, when using hypothetical ligand concentrations estimated from typical abundances of yeast cellular proteins. The ability to compare structurally similar single domain antibodies raised against a single antigen provides a unique opportunity to demonstrate the importance of very low K_{d} to high quality antibody performance in this type of application. Even single domain antibodies with K_{d}s around 10 nM, typically considered high affinity for an antibody, start displaying a precipitous decline in affinity purification performance. These findings highlight the importance of ultra-high affinity reagents, such as the single domain antibodies described here, for the high quality affinity captures required for proteomic and interactomic studies.

Affinity capture experiments were also performed on GFP-tagged Rbm7, a component of the human nuclear exosome, from HeLa cells. Many single domain antibodies demonstrated strong specificity for the targeted complex, comparable to performances seen with yeast-derived Nup84-GFP. However, differences in the amount of contaminants were seen for certain LaGs, notably LaG-41, from purifications in yeast versus HeLa cells, despite high affinity for GFP (K_{d}=0.9 nM) and the efficient recovery of both tagged complexes. These results underscore how even high affinity reagents can give unpredictable background in certain cell types, demonstrating the utility of obtaining large repertoires of such affinity reagents so that at least one is likely to be optimal for any particular application. Similarly, Dynabead-conjugated LaMs were used to isolate mCherry-tagged histone H2B from yeast. For all six LaMs tested, the core nucleosome complex was efficiently isolated, demonstrating the affinity and specificity of this second group of single domain antibodies. Consistent with the low K_{d}s of all the identified LaMs, the yield and specificity of all affinity isolations were similarly high. Commercial RFP-Trap® single domain antibody (ChromoTek GmbH) was also tested in this experiment, and the overall yields were substantially lower.

To test their effectiveness in subcellular localizations, immunofluorescence microscopy was performed with a selection of the LaG repertoire. For a relatively low-abundance target protein with distinct subcellular distributions, we chose GFP-tagged PRC1, a protein associated with the nucleus and microtubules in interphase and the mitotic spindle in mitosis Tissue culture cells stably transfected with PRC1-GFP were fixed, and stained with a small subset of the lowest K_{d} single domain antibodies conjugated to Alexa Fluor® 568. All gave similar specific localization, with a particularly strong signal from LaG-16. This demonstrates that single domain antibodies can prove effective in immunofluorescence microscopy, and it is thus likely that these reagents will prove useful in super-resolution microscopy studies. We also compared the fluorescence spectra of GFP in the presence or absence of various LaGs to look for spectral shifts upon binding, as have previously been reported, and observed moderate increases in fluorescence for several LaGs, with a maximum increase in fluorescence intensity of approximately 60%.

One additional question of specificity analyzed was the ability of a pool of single domain antibodies to recognize other fluorescent homologs of Aequorea victoria GFP and Discosoma mCherry. We tested the 13 highest affinity LaGs against a variety of fluorescent proteins: eGFP, two YFP variants, two CFP variants, BFP, mCherry, and DsRed. As expected, we found that none of these single domain antibodies bound DsRed or mCherry, two Discosoma sp. -derived proteins with low sequence identity to eGFP (<30%), or TurboYFP, derived from Phialidium sp., which has 53% sequence identity to eGFP. All bound standard Aequorea victoria-derived CFP, YFP, and BFP variants (>96% eGFP identity). Interestingly, two LaGs did not bind a moderately divergent (78% eGFP identity) CFP sequence from Aequorea macrodactyla, while all others did. These results indicate that while identified LaGs bind specifically to fluorescent proteins with high identity to eGFP, differential binding activities can be obtained through selection of variants from other species. Anti-mCherry LaM single domain antibodies bound to mCherry, but not to any form of GFP, YFP, or CFP tested. Interestingly, two LaMs (LaM-3 and LaM-4) bound to standard DsRed, from which mRFP1 and mCherry are derived. DsRed has approximately 80% sequence identity to mCherry, and is not recognized by the commercially available RFP-Trap® single domain antibody. Given the different fluorescent protein affinities observed with the LaG and LaM nanobodies, including specificity for AmCFP and DsRed, these reagents have diverse potential uses in differential labeling and affinity capture experiments from cells simultaneously expressing different fluorescently-tagged proteins.

### Mapping of the Single Domain Antibody-Binding Epitopes on GFP

We identified the epitopes on GFP recognized by the twelve highest affinity LaGs using chemical shift perturbation, a well-established nuclear magnetic resonance (NMR) technique. This method allows the mapping of binding sites on a protein by following changes in its characteristic “fingerprint” spectrum (typically the ¹⁵N—¹H HSQC) occurring as a result of adding an unlabeled ligand into a ¹⁵N-labeled protein sample.

Because previous studies have already made backbone ¹⁵N—¹H chemical shift assignments of the GFPuv variant (closely related to standard eGFP with 97% sequence identity), we prepared ¹⁵N-labeled GFPuv, measured its ¹⁵N—¹H HSQC spectrum and obtained the ¹⁵N—¹H chemical shift assignments based on those published. We then prepared complexes between 12 high affinity LaGs and ¹⁵N-labeled GFPuv and measured their ¹⁵N—¹H HSQC spectra. For 11 out of the 12 cases, we observed clear and specific changes in chemical shifts of a large percentage of cross-peaks compared to the ¹⁵N—¹H HSQC spectrum of GFPuv alone. In the 12^{th} case, LaG-24, the single domain antibody did not bind the GFPuv variant. Therefore, we conclude that LaG-24 binds on the face of GFP containing residues S99, T153 and A163—those mutated to obtain GFPuv. This conclusion was supported by the chemical shift assignment we were able to obtain for eGFP (data not shown).

A chemical shift difference was calculated for all spectra, and residues exhibiting a difference higher than 0.03 ppm were judged to be in the binding interface. All the identified epitopes corresponded to large interfaces comprising more than 50 amino acids, consistent with the high affinity binding observed. The binding epitopes of the single domain antibodies can be divided into 3 distinct groups, with closely overlapping epitopes for all single domain antibodies in each group. The binding site of group I, containing 5 single domain antibodies (LaG-16, LaG-9, LaG-14, LaG-43 and LaG-17) overlaps with the binding site of group II, also containing 5 single domain antibodies (LaG-19, LaG-21, LaG-26, LaG-27 and LaG-41), whereas the two group III single domain antibodies (LaG-2 and LaG-24) exhibit a binding epitope on the opposite side of the GFP molecule compared to groups I and II. As a control, we also used this NMR approach to determine the GFPuv binding site of the commercial GFP-Trap® single domain antibody, the structure of whose complex with GFP has been crystallographically determined (PDB ID 3K1K), and showed that the NMR-mapped epitope matched the published results. Comparing the binding epitopes of single domain antibodies with that of GFP-Trap®, group I shows virtually no overlap with the GFP-Trap® binding site, group II has some small overlap, while group III, which binds on the same face of GFP, shows significant overlap.

### Dimerized LaGs as Ultra-High Affinity Reagents

As NMR identified multiple epitopes for these 12 LaGs, we engineered heterodimers of LaGs with non-overlapping binding sites on GFP that could potentially bind with higher affinity. Pairs of LaGs from different binding site groups were genetically fused with different peptide linkers and recombinantly expressed. A LaG16-LaG2 fusion with a flexible glycine-rich linker showed the highest affinity by SPR, with a K_{d} of 36 pM, approximately twenty-fold lower than either LaG alone. Notably, the off-rate of these dimers from GFP, expected to be the major difference in such bivalent binders, decreased almost ten-fold. Dimers of other LaGs or with a different linker (a /3xFLAG tag), displayed K_{d}s in the range of 100-200 pM. We also sought to determine whether the higher affinity of these dimers could result in significantly faster affinity isolations after conjugation to magnetic beads, compared to single single domain antibodies or polyclonal anti-GFP. We therefore performed time courses of yeast Nup84-GFP isolations and compared the relative yields of known Nup84 complex components. The LaG16-LaG2 dimer showed notably higher yields at earlier time points, reaching approximately 90% of maximum yield after only 10 minutes, and 80% after 5 minutes. Thus, these picomolar affinity reagents open the door for increasingly rapid affinity isolations, potentially allowing the capture of weakly or transiently associated complex components for interactome studies. In addition, their high avidity would allow for the detection of low abundance or trace antigens, such as is required for many diagnostic applications.

It will be apparent from the foregoing that the present disclosure provides for the production and generation of single domain antibodies a n d allows for the rapid generation of a large antibody repertoire against multiple epitopes in a chosen antigen. Notably, this approach identifies single domain antibody sequences directly from the source, animal serum. This takes advantage of the complex, natural selection processes occurring in the animal's immune system, avoiding intermediary expression systems, which we couple with non-naturally occurring expression systems to provide recombinant antibodies, including the cell cultures comprising the expression vectors and the expression vectors themselves. Thus, the disclosure ultimately allows for the facile and low-cost production of a comprehensive set of specific high affinity single domain antibodies for use in the isolation and characterization of target macromolecules, such as the GFP-tagged proteins shown here. GFP is one of the most widely used protein tags across all biomedical disciplines, in applications ranging from visualization to proteomics. The enormous number of existing strains and prior research making use of GFP-tagged proteins means that the described improved reagents for the affinity isolation of this tag will be of immediate general use. The disclosure is well-suited to the development of new single domain antibody reagents against various types of protein targets. For example, we have expanded the GFP approach using mCherry as described further herein. In addition to commonly used protein tags, difficult to tag proteins will also be used as antigens to generate directly targeted single domain antibodies. For example, many categories of viral proteins have proven resistant to standard genetic tagging techniques, and are prime candidates for single domain antibody development, in applications from proteomics to therapeutics and diagnostics. Single domain antibodies are much smaller than antibodies, resistant to aggregation, and can be readily humanized. They have great potential in drug development, as they can bind with great specificity and efficacy to disease targets such as tumor cells, either independently (as a monomer or an ultra-high affinity single domain antibody dimer), or as a fusion with other protein domains, molecules, or drugs. Single domain antibodies have proven extremely successful in trials as both potential cancer diagnostics and cancer therapeutics. As demonstrated here, the ability of the presently described methods to quickly and easily identify large repertoires of high affinity bacterially-expressed single domain antibodies against a chosen target antigen has the potential to significantly advance the field.

The following materials and methods illustrate techniques used to obtain results described herein, and include a discussion of, for example,

### Isolation of V_{H}H Antibodies

Llamas were immunized with recombinant GFP-His₆, or recombinant mCherry-His₆ through a subcutaneous injection of 5 mg of protein with CFA. Three additional injections of 5 mg protein, with IFA, were performed at three week intervals. Serum bleeds were obtained 10 days after the final injection. 2.5 ml of serum was diluted ten-fold in 20 mM sodium phosphate, pH 7.0, and incubated with Protein G-agarose resin for 30 min. The flow-through was then incubated for 30 min with Protein A-agarose resin. Both resins were washed with 20 mM sodium phosphate, pH 7.0, and bound VHH IgG was eluted with 100 mM acetic acid, pH 4.0 and 500 mM NaCl (Protein G resin) or 100 mM acetic acid, pH 3.5 and 150 mM NaCl (Protein A resin). These elutions were pooled and dialyzed into PBS. 3 mg of this VHH fraction was then incubated with Sepharose-conjugated GFP. This resin was washed with 10 mM sodium phosphate, pH 7.4 and 500 mM NaCl, followed by 1-4.5 M MgCl₂ in 20 mM Tris, pH 7.5, and then equilibrated in PBS. The resin was then digested with 0.3 mg/ml papain in PBS plus 10 mM cysteine, for 4 hours at 37° C. The resin was then washed with 1) 10 mM sodium phosphate, pH 7.4 and 500 mM NaCl 2) PBS plus 0.1% Tween-20 3) PBS 4) 0.1 M NH₄OAc, 0.1 mM MgCl₂, 0.02% Tween-20. Bound protein was then eluted for 20 min with 0.1 M NH₄OH and 0.5mM EDTA, pH 8.0. These elutions were dried down in a SpeedVac and resuspended in LDS plus 2.5 mM DTT. The samples were alkylated with iodoacetamide and run on a 4-12% Bis-Tris gel. The ˜15 kDa band corresponding to the digested VHH region was then cut out and prepared for MS.

### RT-PCR and DNA Sequencing

Bone marrow aspirates were obtained from immunized llamas concurrent with serum bleeds. Bone marrow plasma cells were isolated on a Ficoll gradient using Ficoll-Paque (GE Healthcare). RNA was isolated from approximately 1-6×10⁷ cells using Trizol LS reagent (Life Technologies), according to the manufacturer's instructions. cDNA was reverse-transcribed using Ambion RETROscript (Life Technologies). A nested PCR was then performed with IgG specific primers. In the first step, CALL001 (5′-GTCCTGGCTGCTCTTCTACAAGG-3′ SEQ ID NO:1) and CALL002 (5′-GGTACGTGCTGTTGA ACTGTTCC-3 SEQ ID NO:2′) primers were used to amplify the IgG variable domain into the CH2 domain. The approximately 600-750 bp band from VHH variants lacking a CH1 domain was purified on an agarose gel. Next, for 454 sequencing, VHH regions were specifically reamplified using framework 1- and 4-specific primers with 5′ 454 adaptor sequences: and 454-VHH-reverse (5′-CTATGCGCCTTGCCAGCCCGCTCAG GGAGACGGTGACCTGGGT-3′ (SEQ ID NO:9)). The approximately 400 bp product of this reaction was gel purified, then sequenced on a 454 GS FLX system after emPCR amplification, on one Pico Titer Plate. For Illumina MiSeq sequencing, the second PCR was instead performed with random 12-mers replacing adaptor sequences, to aid in cluster identification:

MiSeq-VHH-forward
(5′-NNNNNNNNNNNNATGGCT[C/G]A[G/T]GTGCAGCTGGTGGAGT
CTGG-3′ SEQ ID NO: 10))
and
MiSeq-VHH-reverse
(5′-NNNNNNNNNNNNGGAGACGGTGACCTGGGT-3′ SEQ ID
NO: 11)).

The product of this PCR was gel purified, ligated to MiSeq adaptors before library preparation using Illumina kits, and run on a MiSeq sequencer with 2×300 bp paired end reads.

### Database Preparation

The protein sequence databases used for identification were prepared by translating sequencing reads in all 6 reading frames, and for each read the longest Open Reading Frame (ORF) was selected. The selected ORF was digested with trypsin in silico and a list of unique tryptic peptides of 7 amino acids or longer was constructed and saved in a FASTA file. It is important to construct a FASTA file only containing unique peptides because even though most search engines can handle some sequence redundancy, they are not well equipped to handle the extreme redundancy that is provided by next generation sequencing of the single chain antibody locus and search engines either become very slow or crash if presented with such an extreme redundancy.

### Mass Spectrometry

Gel sections containing V_{H}H domains were excised, reduced with DTT (100 μL; 10 mM DTT, 100 mM ammonium bicarbonate) at 56° C. for 30 min, and alkylated with iodoacetamide (100 μL; 55 mM iodoacetamide, 100 mM ammonium bicarbonate) at 25° C. for 20 min in the dark. The dehydrated gel slices were then subjected to in-gel digestion with proteomic-grade trypsin (80 μL; 25 ng trypsin, 25 mM ammonium bicarbonate) (Promega) at 37° C. overnight. The gel was extracted once with extraction solution (140 μL; 67% acetonitrile, 1.7% formic acid). The resulting proteolytic digest was cleaned with a STAGE tip² and loaded onto a home-packed reverse phase C18 column (75 μm I.D., 15 μm tip) (New Objective) with a pressurized bomb. The loaded peptides were subsequently separated with a linear gradient (0% to 42% acetonitrile, 0.5% acetic acid, 120 min, 150 nL/min after flow splitting) generated by an Agilent 1260 HPLC and directly sprayed into an LTQ-Velos-Orbitrap mass spectrometer (Thermo Scientific) for analysis. In the mass spectrometer, a survey scan was carried out in the orbitrap (resolution=30,000, AGC target=1E6) followed by tandem MS in the ion trap (AGC target=5E3) of the top twenty most intense peaks.

Tandem MS was carried out with collision induced dissociation (isolation width=2 Th, CE=35%, activation time=5 ms). Internal calibration was used for improved mass accuracy (lock mass m/z=371.1012). In order to scan more peptides, both predictive AGC and dynamic exclusion were enabled (Repeat counts: 2, repeat duration: 12 s, exclusion duration: 60 s). Single and unassigned charge species were excluded from tandem MS scans. The raw files were converted into mzXML format with ReAdW (version 4.3.1).

### MS-Based Identification of VIM Sequences

The MS search was performed on the custom database of tryptic peptides using the X! Tandem search engine. Then, the identified peptides filtered by expectation value were mapped to the sequences translated from 454 reads (longest ORF only, as described above). The CDR regions were located within the sequence based on approximate position in the sequence and the presence of specific leading and trailing amino acids. For example, to locate the CDR3 region, the algorithm searched for the left anchor YXC (X representing any amino acid) between position 93 and 103 of the sequence, and the right anchor WG between position n-14 and n-4 of the sequence, where n is the length of the sequence. Once the peptides were mapped to the sequences and their CDR regions, a metric was calculated to rank each sequence as a potential candidate based on the bioinformatics evidence available. The factors included in the metric were: MS coverage and length of individual CDR regions with CDR3 carrying highest weight, overall coverage including framework region, and a count of the 454 reads producing the sequence. Finally, sequences with similar CDR3 regions were grouped together, allowing for the identification of the highest confidence sequence corresponding to a particular CDR3. A sequence was assigned to a group where its hamming distance to an existing member was 1, i.e. there was one amino acid difference in the sequence, and different groups that have one shared sequence were further combined. By choosing sequence hits from different groups for production, we maximized the overall sequence diversity of the candidate pool. The candidate list was displayed for manual inspection as an interactive HTML page with CDR regions annotated, peptide mapping information and the ranking metrics shown for each sequence. All algorithms described above were implemented in Perl.

The pipeline that was used for identification of the single domain antibody sequences has been automated and is accessed through a web-based interface which allows upload of FASTA files containing reads from High-throughput DNA sequencing. Once uploaded, the reads will be automatically translated and digested to create an MS searchable database of tryptic peptides, as described above. Next, the MS (mgf) files can be uploaded for a selected tryptic peptide sequence database, and the parent and fragment error can be chosen for the X! Tandem search. Once the mgf files are uploaded, the X! Tandem search will be executed and the matching peptides saved. Then (1) annotation of CDR regions, (2) mapping of the identified peptides and (3) ranking and grouping of candidates are performed automatically, producing an interactive display of the candidate list showing detailed information regarding each sequence and its corresponding rank. Llama-Magic is implemented in Perl, HTML and JavaScript. Manual inspection was performed to make sure a) long CDR3 peptides, which embrace both variable regions and framework regions, have fragmentation pattern within the variable regions; b) CDR3 peptides are unique enough (uniqueness score<100);

### Cloning

Single domain antibody sequences were codon-optimized for expression in E. coli and cloned into pCR2.1 after gene synthesis (Eurofins MWG Operon), incorporating BamHI and XhoI restriction sites at 5′ and 3′ ends, respectively. A pelB leader sequence was cloned into pET21b at NdeI and BamHI restriction sites using complementary primers: 5′-TATGAAATACTTATTGCCTACGGCAGCCGCTGGATTGTTATTACTCGCGGCCCAGC CGGCC ATGGCTG-3′ (SEQ ID NO:12) and 5′-GATCCAGCCATGGCCGGCTGGGCCGCGAGTAATAACAATCCAGCGGCTGCCGTAG GCAA-TAAGTATTTCA-3′ (SEQ ID NO:13). Single domain antibody sequences were then subcloned into pET21b-pelB using BamHI and Xhol restriction sites, with primers also encoding a PreScission Protease cleavage site just before the C-terminal 6xHis tag.

### Purification of Nanobodies

pelB-fused single domain antibodies were expressed under a T7 promoter in Arctic Express (DE3) cells (Agilent), induced with IPTG at a final concentration of 0.1 mM. Cells were induced for 18-20 hours at 12° C., then pelleted by a 10 min spin at 5000×g. The periplasmic fraction was then isolated by osmotic shock. This fraction was bound to His-Select nickel affinity resin (Sigma), washed with His wash buffer (20 mM sodium phosphate pH 8.0, 1 M NaCl, 20 mM imidazole), and eluted with His elution buffer (20 mM sodium phosphate pH 8.0, 0.5 MI NaCl, 0.3 M imidazole). The elution was then dialyzed into PBS.

### Fluorescent Protein Binding Assays

2 μg of fluorescent protein was added to 50 μl of 2 mg/ml E. coli lysate diluted in binding buffer (20 mM HEPES, pH 7.4, 350 mM NaCl, 0.01% Tween-20, 0.1 M PMSF, 3 μg/ml pepstatin A). This was incubated with 25 μl of single domain antibody-Dynabead slurry. After a 30 minute incubation at 4° C., beads were washed with binding buffer and bound protein was eluted with 15 μl LDS. Elutions were run on a 4-12% Bis-Tris gel.

### K_{d} Determinations

SPR measurements were obtained on a Proteon XPR36 Protein Interaction Array System (Bio-Rad). Recombinant GFP or mCherry was immobilized on a ProteOn GLC sensor chip: the chip surface was first activated with 50 mM sulfo-NHS and 50 mM EDC, run at a flow-rate of 30 μl/min for 300 sec. The ligand was then diluted to 5 μg/ml in 10 mM sodium acetate, pH 5.0, and injected at 25 μl/min for 180 sec. Finally, the surface was deactivated by running 1 M ethanolamine-HCl (pH 8.5) at 30 μl/min for 300 sec. This led to immobilization of approximately 600-800 response units (RU) of ligand. K_{d}s of recombinant single domain antibodies were determined by injecting 4 or 5 concentrations of each protein, in triplicate, with a running buffer of 20 mM HEPES, pH 8.0/150 mM NaCl/0.01% Tween. Proteins were injected at 50 μl/min for 120 sec, or 100 μl/min for 90 sec, followed by a dissociation time of 600 sec. Between injections, residual bound protein was eliminated by regeneration with 4.5 M MgCl₂ in 10 mM Tris, pH 7.5, run at 100 μl/min for 36 sec. Binding sensorgrams from these injections were processed and analyzed using the ProteOn Manager software. Binding curves were fit to the data with a Langmuir model, using grouped kₐ, k_{d}, and Rₘₐₓ values.

### Cell Culture and Fluorescence Microscopy

A stable GFP-PRC1 cell line in hTERT-RPE1 cells was cultured on coverslips in DMEM/F-12 media with 10% FBS and penicillin/streptomycin at 37° C. with 8% CO₂ in a humidified environment. For immunofluorescence microscopy, cells were fixed in ice-cold methanol for 10 minutes. After blocking for 30 min with 1% FBS in PBS, the cells were incubated for 1 hour at room temperature with recombinant single domain antibody conjugated to Alexa Fluor 568 succinimidyl ester (Life Technologies), diluted to 5 μg/ml in PBS/1% FBS. Cells were washed with PBS/1% FBS, then mounted with ProLong Gold (Life Technologies).

### Affinity Isolations of Tagged Protein Complexes

Recombinant single domain antibodies were conjugated to epoxy-activated magnetic Dynabeads (Life Technologies), with minor modifications to published IgG coupling conditions. 10 μg recombinant protein was used per 1 mg of Dynabeads, with conjugations carried out in 0.1 M sodium phosphate, pH 8.0 and 1 M ammonium sulfate, with an 18-20 hour incubation at 30° C. Affinity isolations of yeast Nup84-GFP were carried out as previously described, using binding buffer consisting of 20 mM HEPES, pH 7.4, 500 mM NaCl, 2 mM MgCl₂, 0.1% CHAPS, 0.1M PMSF, and 3 μg/ml pepstatin A. For each experiment, 50 μl of bead slurry was used with 0.5 g of yeast cells. Similar conditions were used for HTB2-mCherry isolations (from yeast with HTB2 genomically tagged at the C-terminus with mCherry), except lysate was sonicated 4 times for 10 s before centrifugation, and the binding buffer consisted of 20 mM HEPES, pH 8.0, 300 mM NaCl, 110 mM KOAc, 0.1% Tween-20, 0.1% Triton X-100, 0.1M PMSF, and 3 μg/ml pepstatin A. Isolations of RBM7-LAP from HeLa cells were performed as previously described. 10 μl of bead slurry was used with 100 mg of cells, using a binding buffer of 20 mM HEPES, pH 7.4, 300 mM NaCl, 0.5% Triton X-100, with cOmplete Protease Inhibitor, EDTA-free (Roche).

### Fluorescence Spectra

Samples of recombinant GFP at 0.5 μM in PBS were mixed with either buffer or 10 μM of a LaG protein. Fluorescence spectra were obtained on a Synergy Neo (BioTek) microplate reader. Excitation spectra from 300 nm to 530 nm were taken at an emission wavelength of 560 nm, and emission spectra were measured from 450 nm to 600 nm at an excitation wavelength of 425 nm.

### Phylogenetic Analysis

Phylogenetic trees and alignments were generated from LaG amino acid sequences using the Phylogeny.fr web service.

The following description provides an illustrative and non-limiting protocol for performing embodiments of this disclosure:

### Camelid Immunizations (50-80 Days)

1) Prepare llama or alpaca animals for immunization with the purified antigen(s) of choice. 2) Immunize animals subcutaneously with a 1:1 mixture of Complete Freund's Adjuvant (CFA) and the antigen solution (0.1 to 5 mg of each antigen, depending on anticipated immunogenicity). 3) At 21 day intervals, administer three booster immunizations with a 1:1 mixture of Incomplete Freund's Adjuvant (IFA) and antigen (0.1 to 5 mg) (the number of booster immunizations can be varied). 4) 10 days after final booster, collect production serum bleeds (100 ml or more), and bone marrow aspirates (enough for 5 to 30 ml) from each animal.

### Isolation of HCAb Fraction from Serum (1 day)

5) Dilute 7.5 ml llama serum 1:9 with 67.5 ml 20mM sodium phosphate, pH 7.0, filter with 0.22-μm filter 6) Add to 13 ml Protein G-agarose resin (Invitrogen 10-1243), equilibrated in 5 vol (65 ml) 20 mM sodium phosphate, pH 7.0. Incubate 30 min at room temperature on rotator. 7) Allow Protein G column to drain by gravity and collect flow-through. Then wash with 5 vol (65 ml) 20 mM sodium phosphate, pH 7.0. 8) Add Protein G flow-through to 13 ml Protein A-agarose resin (Invitrogen 10-1042), equilibrated in 5 vol (65 ml) 20 mM sodium phosphate, pH 7.0. Incubate 30 min at room temperature on rotator. 9) Collect flow-through from Protein A column. Wash with 5 vol (65 ml) 20 mM sodium phosphate, pH 7.0. 10) Elute from Protein G resin with 50 ml 100 mM acetic acid, pH 4.0/500 mM NaCl, and immediately neutralize with 5 ml 1 M Tris-HCl, pH 8.0. 11) Elute from Protein A resin with 50 ml 100 mM acetic acid, pH 3.5/150 mM NaCl, immediately neutralize with 5 ml 1 M Tris-HCl, pH 8.0. 12) Pool Protein A and Protein G elutions, and dialyze overnight into 4 L 1×PBS (4×1 L buffer changes, e.g. at 2, 2, 4, and 16 hr intervals) at 4° C. 13)

Concentrate elutions to approximately 50 ml (typically 1-5 mg/ml), using 30 kDa MWCO Amicon Ultra-15 Centrifugal Filtration Units (Millipore), centrifuged at 5,000×g in a table-top centrifuge with swinging bucket rotor. Determine concentration by Bradford or BCA assay, then add 25 μl of 10 mg/ml Protein M-sepharose per mg of HCAb. Incubate for 30 minutes rotating, then collect flow-through.

HCAb affinity purification (˜2 hrs) 14) Conjugate antigen to Dynabeads M-270 epoxy (Life Technologies) according to manufacturer's instructions. Other types of magnetic beads, or CNBr-activated Sepharose can also be used. For Dynabeads, standard conditions for the conjugation use 10 μg of antigen per mg of beads, conjugated in 1 M ammonium sulfate and 0.1 M sodium phosphate, pH 8.0. After a 20-24 hr conjugation at 30° C., beads are washed with quick successive washes in 0.2 M glycine-HCl, pH 2.5, 10 mM Tris-HCl, pH 8.8, 100 mM triethylamine, and 1×PBS. This is followed by four 5 min washes with 1×PBS, a 5 min wash with 1×PBS +0.5% Triton, and a 15 min wash with 1×PBS+0.5% Triton. 15) Add 2-10 mg of VHH IgG to 25-50 mg Dynabeads (or ˜100 u1 of sepharose resin). Amounts can be scaled depending on size of antigen and strength of immune in an effort to obtain 1 μg or more of purified VHH. 16) Incubate 1 hr at room temperature on nutator. 17) Use a magnetic rack to collect beads, and remove VHH IgG solution. Wash beads 2× with 1 ml PBS+0.35M NaCl, 2× with 1 ml 2-4M MgCl₂ (depending on desired stringency), 2× with 1 ml PBS+0.5% Triton X-100, and 2× with 1 ml PBS.

Optional digest of purified HCAb (1 day) 18) Resuspend beads in 200 μl digest solution of a) 2 U/μl IdeS (Genovis FabRICATOR) in PBS (preferred for higher specifity) or b) 0.3 mg/ml papain (Sigma) in PBS/5 mM cysteine. 19) Incubate 4 hrs at 37° C., shaking at 1200 rpm on a Thermomixer. 20) Collect beads using a magnetic rack, and aspirate off the digest solution. 21) Wash twice with 1 ml PBS+0.1% Tween 22) Wash twice with 1 ml last wash buffer (0.1M NH4OAc, pH 7.4/0.1 mM MgCl2/0.02% Tween) 23) To elute, resuspend beads in 0.5 ml elution buffer (0.1M NH4OH/10mM EDTA, pH 8.0). Incubate 20 min at room temperature on rotator. 24) Collect elution from beads, repeat with an additional 0.5 ml. 25) Combine elutions and dry down in a Savant SpeedVac Concentrator. Alternatively, elute directl with 35 μl LDS loading buffer, incubating at 75° C. for 10 min. 26) Resuspend samples in LDS loading buffer with 25 mM DTT (or add DTT to LDS elution to 25 mM), then heat at 75° C. for 10 min. Add 0.1M iodoacetamide and incubate 30 min. at room temperature in the dark. Run on 4-12% Bis-Tris SDS-PAGE polyacrylamide gel (NuPAGE Novex, Life Technologies). Run at 200V for ˜35 minutes with IVIES running buffer. 27) Fix and stain gels for 15 minutes in 0.5% Coomassie Brilliant Blue R-250/45% methanol/10% acetic acid. Destain in 16% methanol/10% acetic acid, changing destain solution three times after 15 minute incubations, or until fully destained.

Mass Spectrometry of VHH IgG (2 days) 28) Excise gel sections containing VHH domains (at 14-30 kDa): Use an ophthalmic standard incision micro scalpel (FEATHER; PFM Medical) to cut the gel band into ˜1 mm×1 mm pieces. Maintain reagents in a clean hood equipped with an air filter (e.g. AirClean 600) to avoid keratin contamination. 29) Destain the gel pieces in destaining solution (2×200 μL; 50% acetonitrile, 50 mM NH4HCO3) by shaking at 1500 rpm at 4° C. for 1 h. 30) Add pure acetonitrile (200 μL) to dehydrate the gel. The gel pieces turn white. Dry the gel pieces in a Savant SpeedVac Concentrator. 31) Hydrate the dried gel pieces with 20 μL sequencing grade trypsin solution (12.5 ng/μL in 25 mM NH4HCO3; Promega). The gel pieces turn transparent again. Cover the gel with an additional 60 μL of 25 mM NH4HCO3. Digest at 37° C. overnight, without shaking. 32) Remove and save the digestion solution in an Eppendorf tube at 4° C. Extract the gel pieces with extraction solution (2×70 μL 67% acetonitrile, 1.7% formic acid) by shaking at 4° C. for 1 h. The gel should shrink significantly. 33) Combine the digestion solution and extraction solution. Dry down to <50 μL in a SpeedVac Concentrator (it is essential to bring the organic content below 10%). 34) Clean the digest with a home-made C18 StageTip1: Place the tip on the top of a 1.5 mL tube through an adaptor. Sequentially condition it with 50 μL methanol, 50 μL 70% acetonitrile+0.1% TFA, and 2×50 μL 0.1% TFA, by centrifugation at 2000 rpm for 1-2 min. 35) Load the digested peptides onto the tip and centrifuge at 2000 rpm for 5 min. Then wash 2× with 50 uL 0.5% acetic acid. Finally, sequentially elute with 50 μL 40% acetonitrile+0.5% acetic acid and 50 μL 80% acetonitrile+0.5% acetic acid. 36) Dry the eluate in a SpeedVac Concentrator. Resuspend the dried peptides by adding ˜20 μL 0.5% acetic acid. Vortex the solution for 1 min then sonicate in a water sonication bath for 15 min. 37) Load ˜⅓ of the solution on to a home-packed reverse phase C18 column (75 μm I.D., 15 μm tip, 5 cm resin) (New Objective) with a pressurized injection cell at 500 psi. 38) Connect the peptide-loaded C18 column to an HPLC system (e.g. Agilent 1200 series). Adjust the flow-rate through the column to ˜150 nL/min with a flow splitter, and separate the peptides using a linear gradient (0% to 42% acetonitrile, 0.5% acetic acid, 120 min) directly sprayed into an LTQ-Velos-Orbitrap mass spectrometer (Thermo Scientific) for MS analysis. 39) The repetitive analytical cycle typically incorporates a high resolution mass scan in the Orbitrap (resolution=30K) followed by tandem MS scans in the ion trap of the 20 most intense peaks observed in each Orbitrap mass spectrum. Other settings of the mass spectrometer include: Spray voltage: 2-2.5 kV; Transfer capillary temperature: 275° C.; S-lens RF: 30% Dynamic exclusion time: 60 s; MS1 target: 1E6: MS1 maximum injection time: 500 ms; MS2 target: 5000; MS2 maximum injection time: 100 ms; Lock mass: m/z=371.1012; Preview mode: disabled; Charge species rejection: singly charged and unassigned; Minimum intensity for MS2: 1000; Isolation width: 2 Th; CID activation time: 5 ms; Normalized collision energy: 35%. 40) Analyze each sample three times to maximize the analysis depth. Convert all three instrument binary raw files from the same sample into a single MGF file by commonly used software (e.g. MM File Conversion), and upload to the a web server we designed.

Preparation of cDNA from bone marrow aspirates (1 day) 41) Obtain bone marrow aspirates from immunized llamas, taken concurrently with serum bleeds. A 10-30 ml total volume is optimal. 42) Mix 9 parts RPMI 1640 medium plus 10% FBS with 1 part bone marrow aspirate. 43) In a 50 ml Falcon tube(s), overlay 1 part Ficoll-Paque (GE Healthcare) with 2 parts of the diluted aspirate. 44) Centrifuge Ficoll gradient (with brake deactivated) at 800×g for 40 minutes at room temperature (22° C.), in a table-top centrifuge with swinging bucket rotor. 45) Collect interface with lymphocytes and transfer to new tube. 46) Wash cells with 30 ml ice cold RPMI 1640 medium plus 10% FBS, and centrifuge at 300×g for 10 min at 4° C., in a table-top centrifuge with swinging bucket rotor. 47) Count cells with a hemocytometer, and resuspend in TRIzol (Life Technologies), using 1 ml per 1×107 cells. 48) Add 0.2 ml chloroform per 1 ml of TRIzol and shake tubes vigorously for 15 sec. 49) Incubate 3 minutes at room temperature. 50) Centrifuge 12,000×g in microcentrifuge for 15 min at 4° C. 51) Remove upper phase, transfer to new tube. 52) Add 0.5 ml isopropanol per 1 ml TRIzol used. 53) Incubate 10 min at room temperature. 54) Centrifuge at 12,000×g for 10 min at 4° C. 55) Remove supernatant, and wash pellet with 1 ml 75% ethanol per 1 ml TRIzol used. 56) Vortex briefly, and centrifuge 7,500 x g for 5 min at 4° C. 57) Air dry RNA, resuspend in nuclease-free H2O. 58) Reverse transcribe RNA using Ambion RETROscript kit (Life Technologies): add 5 μg of RNA in 9 μl to 2 μl Oligo(dT). 59) Heat at 80° C. for 3 min. 60) Add 2 μl 10×RT buffer, 4 μl dNTP mix, 1 μl RNase inhibitor, and 1 μl MMLV-RT enzyme to reaction. 61) Incubate at 46° C. for 90 min. 62) Incubate at 92° C. for 10 min.

PCR amplification and sequencing of VHH sequences (1 day) 63) Perform the first step of a nested PCR with IgG specific primers CALL001 (5′-GTCCTGGCTGCTCTTCTACAAGG-3′ (SEQ ID NO:1)) and CALL002 (5′-GGTACGTGCTGTTGAACTGTTCC-3′ SEQ ID NO:2): In a 100 μl volume with 1× ThermoPol reaction buffer, 2 mM MgSO4, 0.2 mM dNTPs, and 0.5 μM CALL001 and CALL002 primers, combine 5 μL of the reverse transcription reaction and 2 μL of Deep VentR polymerase. 64) After initial denaturation for 3 min at 94° C., amplify with 30 cycles: denature at 94° C. for 30 sec, anneal at 60° C. for 1 min, and extend at 72° C. for 50 sec. Run a final extension at 72° C. for 10 min. 65) Separate the PCR products by electrophoresis on a 1.2% agarose gel with TBE running buffer. Purify the approximately 600-750 bp band corresponding to VHH variant IgG. 66) With purified DNA as template, PCR amplify again using framework 1- and 4-specific primers. For Illumina MiSeq sequencing, a random 12-mer is added to primers to aid cluster identification: MiSeq-VHH-forward (5′-NNNNNNNNNNNNATGGCT[C/G]A[G/T]GTCGCAGCTGGTGGAGTCTGG-3′ (SEQ 13 NO:10) and MiSeq-VHH-reverse (5′- GGAGACGGTGACCTGGGT-3′ (SEQ NO:11)). In a 100 μl volume with 1× ThermoPol reaction buffer, 2 mM MgSO4, 0.2 mM dNTPs, and 0.5 μM of each primer, combine ˜200 ng of purified product from the first PCR step and 2 μL of Deep VentR polymerase. Repeat the PCR protocol from Step 64. As a preferred alternative to the above PCRs, a single step PCR is done with hinge-specific primers: forward primers 6N_CALL001 5′-NNNNNNGTCCTGGCTGCTCTTCTACAAGG-3′ (SEQ ID NO:1) and 6N_CALL001B 5′-NNNNNNGTCCTGGCTGCTCTTTTACAAGG-3′ (SEQ ID NO:7) and reverse primers 6N_VHH_SH_rev 5′-NNNNNNCTGGGGTCTTCGCTGTGGTGC-3′ (SEQ ID NO:14) and 6N_VHH_LH_rev 5′-NNNNNNGTGGTTGTGGTTTTGGTGTCTTGG G-3′, (SEQ ID NO:15) each including 6 random bases (N). In a 100 μl volume with 1× ThermoPol reaction buffer, 2 mM MgSO4, 0.2 mM dNTPs, 0.5 μM 6N_CALL001, and 0.25 μM each of 6N_VHH_SH and 6N_VHH_LH primers, combine 5 μL of the reverse transcription reaction and 2 μL of Deep VentR polymerase. 67) Separate the PCR product on an agarose gel as in Step 65, and gel purify the ˜400 bp VHH band. 68) For Illumina MiSeq of NovaSeq sequencing, ligate adaptors and generate libraries using Illumina kits, before sequencing with 2×300 bp paired end reads (MiSeq) or 2×250 bp paired end reads (NovaSeq). 69) The high-throughput sequencing data is uploaded in FASTA form to the Llama Magic a web server we developed to generate candidate lists with MS data 70) The software will identify and rank candidate sequences according to sequence coverage and MS/sequencing abundance, so unique sequences can be selected from the top hits on this list.

Cloning and screening recombinant single domain antibodies (˜3-6 days) 71) Candidate sequences can be cloned by gene synthesis, after codon optimization for E. coli. For ease of cloning, in frame BamHI and Xhol restriction sites can be incorporated at 5′ and 3′ ends of the synthesized gene, respectively. 72) Subclone synthesized single domain antibody sequences into pET21b-pelB using BamHI and XhoI restriction sites. 73) To screen for expression and antigen binding, transform pET21b-pelB single domain antibody plasmids into Arctic Express (DE3) cells (Agilent) or BL21(DE3) cells. 74) Grow up 40 mL of transformed cells to OD ˜0.7 and induce with IPTG at a final concentration of 0.1 mM. Induce for 18-20 hours at 12° C. (Arctic Express) or 18° C. (BL21(DE3)). 75) Harvest cells by spinning 10 min at 5000×g at 4° C., remove media, and proceed directly to periplasmic purification. 76) Per 40 mL of original culture volume, resuspend pellet in 0.4 mL ice cold TES (0.2M Tris-HCl, pH 8.0, 0.5 mM EDTA, pH 8.0, 0.5 M sucrose). 77) Per 40 mL of original culture volume, add 0.6 ml of one part TES diluted with 4 parts ddH2O (ice cold). 78) Incubate on ice 30 min. 79) Centrifuge 30 min at 14,000 rpm in a microcentrifuge at 4° C. 80) Collect supernatants, add 110 μL of 10×TBT/NaCl (0.2 M HEPES, pH 7.4, 1.1 M potassium acetate, 20 mM MgC12, 1% Tween-20, 1.5 M NaCl). Save 10 μL for “Input” sample. Other buffers or salt strength can be substituted depending on desired binding affinity or application 81) Equilibrate 2.5 mg of Dynabeads conjugated to target antigen (above) with 1×TBT/NaC1 (20 mM HEPES, pH 7.4, 110 mM potassium acetate, 2 mM MgCl2, 0.1% Tween-20, 0.15 M NaCl). 82) Add supernatants to beads. Incubate for 1 hr, rotating at 4° C. 83) Collect beads on a magnetic rack, collect flow-through and wash 3× with 1 ml 1×TBT/NaCl. Save 10 μL of flow-through for “Flow-through” sample. 84) To elute, resuspend beads in 25 μL of 1×LDS (NuPAGE, Life Technologies). 85) Heat for 10 min at 75° C. Transfer LDS elution to new tube and add DTT to 50 mM. Heat for 10 min at 98° C. 86) Add 1×LDS/50 mM DTT to “Input” and “Flow-through” samples, and heat for 10 min at 98° C. 87) Run input, flow-through, and elution samples on 4-12% Bis-Tris gel (NuPAGE Novex, Life Technologies). Run at 200V for 40 min with MES running buffer. Stain in Coomassie blue (see Step 27), and select those single domain antibodies that express and bind well.

Large-scale purification of recombinant single domain antibodies (2 days) 88) Grow up 1-6 L of cells to OD ˜0.7 and induce with IPTG at a final concentration of 0.1 mM. Induce for 18-20 hours at 12° C. (Arctic Express) or 18° C. (BL21(DE3)). 89) Harvest cells by spinning 10 min at 5,000×g at 4° C., remove media, and proceed directly to periplasmic purification. 90) Per 1 L of original culture volume, resuspend pellet in 10 ml ice cold TES (0.2 M Tris-HCl, pH 8.0, 0.5 mM EDTA, pH 8.0, 0.5 M sucrose). 91) Per 1 L of original culture volume, add 15 ml, ice cold, of one part TES diluted with 4 parts ddH2O (i.e. 3 ml TES plus 12 ml ddH2O). 92) Incubate on ice 30 min. 93) Spin 6000×g, 10 min at 4° C. 94) Take supernatant, spin 48,000×g, 15 min at 4° C. 95) Take supernatant, add 5 M NaC1 to 0.15M. 96) Equilibrate Ni-NTA resin with 5 volumes binding buffer (20 mM Tris-HCl, pH 8.0, 0.15 M NaCl). 97) Incubate periplasmic sample with Ni-NTA resin for 30 min at 4° C. 98) Collect flow-through from column. 99) Wash with 6 column volumes Wash Buffer I (20 mM Tris-HCl, pH 8.0, 0.9 M NaC1). 100) Wash with 6 column volumes Wash Buffer II (20 mM Tris-HCl, pH 8.0, 0.15 M NaCl, 10 mM imidazole-HCl, pH 8.0). 101) Elute with 4 column volumes Elution Buffer (20 mM Tris-HCl, pH 8.0, 0.15 M NaCl, 250 mM imidazole-HCl, pH 8.0). 102) Assess elutions by SDS-PAGE, pool/concentrate as needed, and dialyze into desired buffer. Standard PBS-like buffers are suitable in most cases.

While the invention has been particularly shown and described with reference to specific embodiments (some of which are preferred embodiments), it should be understood by those having skill in the art that various changes in form and detail may be made therein without departing from the spirit and scope of the present invention as disclosed herein.

1. A method for identifying single domain antibodies that bind with specificity to an antigen (heavy chain only IgG class of antibodies (Ag-specific HCAbs)), the method comprising:
i) introducing into a camelid an antigen such that a plurality of Ag-specific HCAbs is produced by the camelid;
ii) testing lymphocytes obtained from the camelid to determine polynucleotide sequences encoding the variable region (V_{H}H) of a mixed population of HCAbs that includes the plurality of Ag-specific HCAbs and HCAbs that are not specific for the antigen (non-specific HCAbs), and deducing the amino acid sequences of the V_{H}H regions of the Ag-specific HCAbs and non-specific HCAbs in the mixed population from the polynucleotide sequences;
iii) processing a sample from the camelid to separate Ag-specific HCAbs from non-specific HCAbs and determining the amino acid sequences of at least a portion of the V_{H}H regions of the Ag-specific HCAbs; and
iv) comparing deduced amino acid sequences of ii) with amino acid sequences of iii) to identify amino acid sequences of ii) that are the same as the amino acid sequences of iii), thereby identifying the Ag-specific V_{H}H regions that are members of the mixed population of HCAbs.

2. The method of claim 1, wherein in step iii) separating the Ag-specific HCAbs comprises removal of non-HCAb antibodies using Protein M, isolating Ag-specific HCAbs, digesting the isolated HCAbs using IdeS protease, separating digested fragments comprising segments of the V_{H}H regions by gel electrophoresis to obtain separated fragments of the V_{H}H regions from the gel, and using the separated segments of the V_{H}H regions to determine the amino acid sequences of at least a portion of the Ag-specific HCAbs.

3. The method of claim 2, wherein the determining the amino acid sequences of iii) comprises mass spectrometric analysis of the Ag-specific V_{H}H regions.

4. The method of claim 3, wherein the determining the polynucleotide sequences of ii) comprises generating and sequencing a plurality of cDNA sequences that encode at least unique V_{H}H regions.

5. The method of claim 3, wherein identifying the deduced amino acid sequences of ii) that are the same as the amino acid sequences of iii) comprises a microprocessor implemented comparison of the amino acid sequences of ii) and iii).

6. The method of claim 3, wherein identifying the deduced amino acid sequences of ii) that are the same as the amino acid sequences of iii) comprises a microprocessor implemented comparison of the measured tandem mass spectra of ii) and the calculated tandem mass spectra of iii).

7. The method of claim 3, wherein separating Ag-specific antibodies from the non-specific antibodies comprises affinity purification of the Ag-specific antibodies using the antigen as an affinity capture agent.

8. The method of claim 3, wherein the lymphocytes of ii) are obtained from bone marrow of the camelid.

9. The method of claim 3, wherein the lymphocytes of ii) comprise B plasma cells.

10. The method of claim 3, wherein the sample of iii) comprises serum from the camelid.

11. The method of claim 3, further comprising providing and introducing distinct expression vectors encoding distinct Ag-specific single domain antibodies into host cells, wherein the single domain antibody sequences are designed based on the deduced Ag-specific V_{H}H regions allowing expression of the distinct Ag-specific single domain antibodies from the host cells, separating the Ag-specific single domain antibodies from the host cells, and testing the Ag-specific single domain antibodies for binding to the antigen.

12. The method of claim 11, wherein the testing the Ag-specific single domain antibodies for binding to the antigen comprises testing for affinity and/or specificity for the antigen.

13. The method of claim 12, wherein the testing comprises identifying one or more of the Ag-specific single domain antibodies that have a Kd in a sub-micromolar range.

14. The method of claim 3, wherein the camelid is selected from camels, alpacas and llamas.


SEQUENCE LISTING (15 sequences)

SEQ ID NO: 1 - DNA, 23 bp, artificial sequence
  primer
gtcctggctgctcttctacaagg

SEQ ID NO: 2 - DNA, 23 bp, artificial sequence
  primer
ggtacgtgctgttgaactgttcc

SEQ ID NO: 3 - DNA, 54 bp, artificial sequence
  primer
  misc_feature (32)..(32): n is a, c, g, or t
  misc_feature (34)..(34): n is a, c, g, or t
cgtatcgcctccctcgcgccatcagatggctnangtgcagctggtggagtctgg

SEQ ID NO: 4 - DNA, 43 bp, artificial sequence
  primer
ctatgcgccttgccagcccgctcagggagacggtgacctgggt

SEQ ID NO: 5 - DNA, 29 bp, artificial sequence
  primer
  misc_feature (1)..(6): n is a, c, g, or t
nnnnnngtcctggctgctcttctacaagg

SEQ ID NO: 6 - DNA, 29 bp, artificial sequence
  primer
  misc_feature (1)..(6): n is a, c, g, or t
nnnnnngtcctggctgctcttttacaagg

SEQ ID NO: 7 - DNA, 27 bp, artificial sequence
  primer
  misc_feature (1)..(6): n is a, c, g, or t
nnnnnnctggggtcttcgctgtggtgc

SEQ ID NO: 8 - DNA, 31 bp, artificial sequence
  primer
  misc_feature (1)..(6): n is a, c, g, or t
nnnnnngtggttgtggttttggtgtcttggg

SEQ ID NO: 9 - DNA, 43 bp, artificial sequence; identical to SEQ ID NO: 4
  primer

SEQ ID NO: 10 - DNA, 41 bp, artificial sequence
  primer
  misc_feature (1)..(12): n is a, c, g, or t
  misc_feature (19)..(19): n is a, c, g, or t
  misc_feature (21)..(21): n is a, c, g, or t
nnnnnnnnnnnnatggctnangtgcagctggtggagtctgg

SEQ ID NO: 11 - DNA, 30 bp, artificial sequence
  primer
  misc_feature (1)..(12): n is a, c, g, or t
nnnnnnnnnnnnggagacggtgacctgggt

SEQ ID NO: 12 - DNA, 68 bp, artificial sequence
  primer
tatgaaatacttattgcctacggcagccgctggattgttattactcgcggcccagccggccatggctg

SEQ ID NO: 13 - DNA, 70 bp, artificial sequence
  primer
gatccagccatggccggctgggccgcgagtaataacaatccagcggctgccgtaggcaataagtatttca

SEQ ID NO: 14 - DNA, 27 bp, artificial sequence
  primer
  misc_feature (1)..(6): n is a, c, g, or t
nnnnnnctggggtcttcgctgtggtgc

SEQ ID NO: 15 - DNA, 31 bp, artificial sequence; identical to SEQ ID NO: 8
  primer
  misc_feature (1)..(6): n is a, c, g, or t